(19) United States
(12) Patent Application Publication
(10) Pub. No.: US 2009/0082315 A1
(43) Pub. Date: Mar. 26, 2009

(54) Compositions and Methods for Controlling Cholesterol Levels

(76) Inventor: Raif Tawakol, Merced, CA (US)

Correspondence Address: STOUT, UXA, BUYAN & MULLINS LLP, 4 VENTURE, SUITE 300, IRVINE, CA, 92618, US

(21) Appl. No.: 12/205,650

(22) Filed: Sep. 5, 2008

Related U.S. Application Data
(63) Continuation-in-part of application No. 11/899,284, filed on Sep. 5, 2007.
(60) Provisional application No. 60/967,797, filed on Sep. 7, 2007.

(51) Int. Cl. A61K 31/455; A61K 31/60

(52) U.S. Cl. 514/161; 514/356

(57) ABSTRACT

New compositions and methods are provided for controlling cholesterol levels. The present compositions are uniquely structured to allow a subject to whom the compositions, in the form of unit dosage forms, are administered to benefit from both a statin, such as atorvastatin, and niacin.

### RELATED APPLICATIONS

This application claims the benefit of and claims priority to U.S. Provisional Application No. 60/967,797, filed Sep. 7, 2007, and is a continuation-in-part of U.S. application Ser. No. 11/899,284, filed Sep. 5, 2007, each of which prior applications are incorporated in their entireties herein by reference.

### BACKGROUND OF THE INVENTION

The present invention is related to compositions and methods for controlling cholesterol levels in subjects, for example, humans and animals. More particularly, the invention relates to compositions and methods for controlling both HDL, (high density lipoprotein) and LDL (low density lipoprotein).

A cluster of inter-related plasma lipid and lipoprotein abnormalities associated with alterations in HDL (high density lipoprotein) and HDL-2b metabolism contributes to the risk of atherosclerosis and cardiovascular events in patients with insulin resistance and type 2 diabetes. HDL and HDL-2b levels control atherogenesis, vascular inflammation, endothelial function and thrombogenicity. The alteration in particle size of both HDL and LDL (low density lipoprotein) contribute to events and progression of disease. Therefore there is a need in the art for therapies that increase HDL and HDL-2b levels.

A class of compounds, commonly known as statins, are very useful in treating hypercholesterolemia, as well as other diseases/conditions. A number of these compounds are commercially available. One example of such a statin is atorvastatin, sold by Pfizer under the trademark Lipitor®.

Niacin has been used in an attempt to raise HDL levels and to lower very low density lipoprotein (VLDL) triglycerides and LDL levels. When tolerated, it is effective as either a primary therapy or an adjunctive therapy. Numerous side effects limit its use in well over 50% of patients in which it has been tried. These side effects include an intense inflammation, or flushing, and associated itching, or pruritus, that usually involves the face and upper part of the body, often involving the entire body.

Most clinicians are concerned about using or dosing niacin and a statin at the same time because these materials may interact with each other causing each to be less effective and/or resulting in a reduction in the amount and/or quality of HDL present in the blood of the subject, which can be harmful, and even result in the death of the subject. In fact, the U.S. Food and Drug Administration (FDA) has issued a warning regarding the practice of giving both a statin and niacin together, despite the fact that this often occurs.

It would be advantageous to obtain the benefits of a statin and niacin while reducing or even eliminating the risks involved in dosing them together.

### SUMMARY OF THE INVENTION

New compositions and methods for controlling cholesterol levels, for example and without limitation one or more of HDL levels, HDL-2b levels and LDL levels, have been discovered. The present compositions, for example, in the form of dosage forms, such as unit dosage forms, allow one to benefit from both a statin, such as atorvastatin, and niacin, while avoiding the immediate and proximate clash between the statin and niacin. It has been found that the unique structures of the present dosage forms allow a subject, e.g., a human or animal, being treated to obtain substantially the full benefit of both the statin and the niacin present in the dosage forms, for example, in controlling cholesterol levels, such as one or both HDL and LDL levels, substantially without the risks warned of by the FDA. Methods of controlling cholesterol levels in accordance with the present invention comprising administering, for example and without limitation, orally administering, the present dosage forms are easy and straightforward to practice, and are effective in providing acceptable therapeutic effects, for example and without limitation, controlled cholesterol levels.

In one broad aspect, the present invention provides one or more dosage forms, such as unit dosage forms, comprising a therapeutically effective amount of a niacin component; and a therapeutically effective amount of an atorvastatin component, with the dosage form or forms being structured so that, when the dosage form is administered to a subject, the niacin component is released from the dosage form prior to the release of the atorvastatin component from the dosage form.

The present invention may employ one or more statin components, such as the commonly known and/or commercially sold compounds known as statins. The invention advantageously employs one or more atorvastatin components. Thus, in many instances throughout this disclosure reference is made to an atorvastatin component. One or more other statins, as the term “statins” is commonly used in the pharmaceutical industry, may be used in combination with or in place of the atorvastatin component. Thus, any and all statins, alone or in combination of two or more statins, may be employed in accordance with the present invention.

In one embodiment, the present dosage forms are structured so that substantially all of the niacin component is released from the dosage form prior to the time substantially any of the atorvastatin component is released from the dosage form. Such totally or entirely separate releases of first the niacin component and, thereafter, the atorvastatin component, from the dosage form allows the atorvastatin component to be effective in the subject to whom the dosage form is administered, for example and without limitation, on such subject's metabolism, separately or at a different time relative to the niacin component. This feature may result in an advantageously increased level of HDL and/or HDL-2b in such subject, for example, relative to the administration of a dosage form in which the same amounts of niacin component and atorvastatin component are present and are released at the same time.

The dosage forms may further comprise a therapeutically effective amount of a non-steroidal anti-inflammatory drug (NSAID) component, for example and without limitation, an aspirin component. In one embodiment, the dosage forms are structured so that, when the dosage form is administered to a subject, the NSAID component is released from the dosage form prior to the release of the atorvastatin component from the dosage form.

The dosage form may be structured so that, when the dosage form is administered to a subject, the niacin component and the non-steroidal anti-inflammatory component are released from the dosage form at substantially the same time and/or over substantially the same period of time.

In one embodiment, the dosage forms may be structured to release the niacin component from the dosage form substantially immediately, for example, within about 1 minute or less, or less than about 10 minutes after the dosage form is administered to a subject. The dosage forms may be structured to release the niacin component from the dosage form for or over a first period of time after the dosage form is administered to a subject. For example, this first period of time may be at least about 10 minutes or about 30 minutes or about 1 hour. This first period of time may be no more than about 3 hours or about 4 hours or about 5 hours or about 6 hours or about 7 hours or about 8 hours or about 10 hours.

In embodiments in which the dosage forms include a NSAID component, the dosage form may be structured to release the NSAID component from the dosage form substantially immediately, as defined elsewhere herein, as or over a second period of time after the dosage form is administered to the subject. This second period of time may be at least about 10 minutes or about 30 minutes or about 1 hour. This second period of time may be no more than about 3 hours or about 4 hours or about 5 hours or about 6 hours or about 7 hours or about 8 hours or about 10 hours.

The dosage forms may be structured to release the niacin component and the NSAID component from the dosage form substantially immediately, as defined elsewhere herein, or for or over substantially the same period of time after the dosage form is administered to the subject.

In one useful embodiment, the dosage forms further comprise a therapeutically effective amount of an additional niacin component, separate from the niacin component noted elsewhere herein. The dosage forms may be structured to release the niacin component before releasing the additional niacin component. For example, the dosage forms may be structured to release the niacin component up to about 1 hour or about 2 hours or about 3 hours or about 4 hours or about 5 hours or about 6 hours or about 7 hours or about 8 hours or about 10 hours prior to releasing the additional niacin component. In one embodiment, the dosage forms are structured to release the niacin component and the additional niacin component prior to releasing the atorvastatin component, for example, prior to the time substantially any of the atorvastatin component is released from the dosage form.

The dosage forms may be structured so that the release of the atorvastatin component from the dosage form, after the dosage form is administered to a subject, is delayed for at least about 10 minutes or about 30 minutes or about 1 hour or at least about 2 hours or about 3 hours or about 4 hours or about 5 hours or about 6 hours and no more than about 10 hours or about 11 hours or about 12 hours or about 13 hours or about 14 hours or about 15 hours. In one embodiment, the dosage forms are structured so as to delay the release the atorvastatin component from the dosage form for at least about 4 hours or about 5 hours and no more than about 9 hours or about 14 hours after the dosage form is administered to a subject.

In one embodiment, the dosage forms are structured so that, after the dosage form is administered to the subject and after a period, if any, in which the release of the atorvastatin component is delayed, the release of the atorvastatin component from the dosage form is further controlled, for example and without limitation, to obtain a therapeutically desired and/or advantageous atorvastatin component release profile and/or concentration profile in the body of the subject. For example and without limitation, the dosage forms may be structured so that, after the dosage form is administered to the subject and after a period, if any, in which the release of the atorvastatin component is delayed, the atorvastatin component is released from the dosage form substantially immediately, as defined elsewhere herein, or for or over a period of time, for example, of at least about 10 minutes or about 30 minutes or about 1 hour or about 2 hours or about 3 hours or about 4 hours or about 5 hours or about 6 or about 7 hours or about 8 hours or about 9 hours or about 10 hours. In one embodiment, this period of time for or over which the atorvastatin component is released from the dosage form is in a range of about 2 hours to about 8 hours.

In another broad aspect of the present invention one or more dosage forms, for example and without limitation, unit dosage forms, are provided comprising a therapeutically effective amount of a niacin component; a therapeutically effective amount of an atorvastatin component; and a delayed release component, for example and without limitation, substantially surrounding the atorvastatin component. The delayed release component is effective to delay the release of the atorvastatin component from the dosage form. The dosage form may further comprise a controlled release excipient or component operatively coupled to the atorvastatin component and present in an amount effective to control, for example and without limitation, slow or otherwise control, the release of the atorvastatin component from the dosage form, after a period of time in which the release of the atorvastatin component is delayed, relative to an identical or a substantially identical dosage form without the controlled release component.

The delayed release components referred to herein may be, for example and without limitation, enteric components or coatings, other components useful to delay the release of pharmaceuticals, conventional materials used to delay release of pharmaceuticals and the like and mixtures thereof. In one embodiment, the delayed release component may be positioned or located so as to have substantially no effect on the release of the niacin component from the dosage form, for example and without limitation, so that the delayed release component does not substantially surround the niacin component.

In one embodiment, the delayed release component is present in an amount effective to delay the release of the atorvastatin component from the dosage form or forms after the dosage form or forms is orally administered to a human subject relative to an identical or a substantially identical dosage form without the delayed release component.

In one embodiment, the dosage forms may further comprise a therapeutically effective amount of an additional niacin component, separate from the niacin component noted above, and a delayed release component or a second delayed release component positioned or located to delay the release of the additional niacin component relative to the release of the niacin component. The dosage forms may be structured to release the niacin component and the additional niacin component prior to releasing the atorvastatin component, for example and without limitation, to release substantially all of the niacin component and the additional niacin component prior to releasing substantially any of the atorvastatin component.

The present dosage forms including a delayed release component may further comprise a therapeutically effective amount of a NSAID component, for example and without limitation, an aspirin component. In one embodiment, the dosage forms are structured so that, when the dosage form is administered to a subject, the NSAID component is released from the dosage form prior to the release of the atorvastatin component from the dosage form.

In one embodiment, the dosage forms may be provided so that the niacin component and the NSAID component are present in a substantially homogenous mixture and/or in a single layer.

In one embodiment, the dosage forms further comprise an intermediate release excipient or component present in an amount and in a position or location effective to control the release, for example and without limitation, to control the rate of release, of the niacin component, for example, after the administration of the dosage form to a subject.

The present dosage forms may take on any suitable physical form or forms provided that such dosage forms function and are effective, for example and without limitation, as set forth elsewhere herein. In one embodiment, the present dosage forms are solid unit dosage forms.

In a further broad aspect of the present invention, methods of controlling cholesterol levels in a subject are provided. In general, the present methods comprise administering one or more of the dosage forms, as described herein, to a subject, such as an animal subject or a human subject. Such administering may be for a sufficient period of time to thereby control the levels of cholesterol, for example and without limitation, to increase the level of HDL and/or HDL-2b in the subject.

In one embodiment, the present methods comprise administering to the subject, a dosage form or an unit dosage form comprising a therapeutically effective amount of a niacin component, and a therapeutically effective amount of an atorvastatin component, the dosage form being structured so that, after the administering step, the niacin component is released from the dosage form prior to the atorvastatin component being released from the dosage form.

In one embodiment, the present methods comprise administering a dosage form as noted above, which dosage form further comprises a therapeutically effective amount of a NSAID component, for example and without limitation, an aspirin component. The dosage form may be structured so that, after the administering step, the NSAID component is released from the dosage form prior to the atorvastatin component being released from the dosage form. In one embodiment, the dosage form is structured so that, after the administering step, the niacin component and the NSAID component are released from the dosage form at substantially the same time and/or over substantially the same time period.

In one embodiment, the administering step of the present methods comprises administering a dosage form to the subject twice daily. For example and without limitation, a dosage form may be administered to the subject about 30 to about 60 minutes after the subject has eaten a midday meal, such as a lunch meal or lunch, and a dosage form is administered to the subject about 30 minutes to about 60 minutes after the subject has eaten the evening meal, such as a dinner meal or dinner.

During at least a major portion, that is at least about 50%, of the total time period during which a human subject, for example, an adult human subject weighing between about 100 pounds or less or about 120 pounds to about 250 pounds or about 300 pounds or more, is treated with the present dosage forms in accordance with the present invention, the total daily dosage of the niacin component from the administered dosage form or forms may be in the range of about 200 mg or about 375 mg or about 750 mg to about 1000 mg or about 1500 mg or about 2000 mg. During at least a major portion of the total time during which a human subject, for example, as noted above, is treated with the present dosage forms in according with the present invention, the total daily dosage of the atorvastatin component from the administered dosage form or forms may be in a range of about 2 mg or less or about 5 mg or about 10 mg to about 15 mg or about 25 mg or about 35 mg or more. Also, during at least a major portion of the total time period during which a human subject, for example, as noted above, is treated with the present dosage forms in accordance with the present invention, the total daily dosage of the NSAID component from the administered dosage form or forms may be in a range of about 5 mg or less or about 10 mg or about 25 mg to about 50 mg or about 81 mg or about 165 or more.

In one embodiment of the present invention, methods are provided which may provide substantial benefits and involve treatment of a subject, e.g., a human subject, comprising administering dosage forms in accordance with the present invention to the subject twice daily, before lunch and before dinner, as noted elsewhere herein.

In one embodiment, such methods provide that, during a first or initial period of time of the treatment, for example, for the first about 3 to about 10 days of the treatment, such as the first 6 days of treatment, each dosage form administered includes the niacin component in an amount in a range of about 62.5 mg to about 125 mg; during a second period of time, for example, for the next about 5 to about 12 days of treatment, such as the next 8 days of treatment, each dosage form administered includes the niacin component in an amount of about 200 mg to 300 mg, for example and without limitation, about 250 mg, and during a third period of time, for example, during the following about 12 to about 20 days of treatment, each dosage form administered includes the niacin component in a larger amount than in the preceding days of treatment, for example and without limitation, in an amount of about 300 mg to about 750 mg or about 1000 mg, for example and without limitation, about 375 mg.

In one embodiment, during the second and third periods of time noted above, each dosage form administered after lunch includes the niacin component in an amount of about 250 mg. In one embodiment, during the second and third periods of time noted above, each dosage form administered after dinner includes the niacin component in an amount of about 500 mg.

In one embodiment, during the third period of time noted above, each dosage form administered after lunch includes the niacin component in an amount of about 200 mg to about 400 mg, and each dosage form administered after dinner includes a larger amount of the niacin component, for example, about 400 mg to about 750 mg or about 1000 mg of the niacin component.

In one embodiment, during the first period of time of the treatment noted above, each dosage form administered includes the niacin component in an amount in a range of about 62.5 mg to about 125 mg; during the second period of time of treatment each dosage form administered includes the niacin component in an amount of about 375 mg; and thereafter during the treatment, for example and without limitation, during the third period of time of treatment noted above, each dosage form administered includes the niacin component in an amount of about 750 mg.

In one embodiment, after the three periods of time of treatment noted above, if the treatment is to continue, each dosage form administered may include the niacin component in an amount of about 500 mg to about 750 mg.

In one embodiment, the present methods comprise administering two dosage forms, such as dosage forms in accordance with the present invention, comprising a niacin component and an atorvastatin component daily to a subject, such as a human subject.

In one embodiment, the present dosage forms may not be administered to a subject in the morning, before or after the first meal of the day, such as breakfast, or before lunch.

In one embodiment, the first daily dose, for example, to be administered shortly after lunch as described elsewhere herein, includes one-half of the total daily dose of niacin component and one-half of the total daily dose of the atorvastatin component. The second daily dose, for example, to be taken shortly after dinner as described elsewhere herein, includes the other half of the daily dose of both the niacin component and the atorvastatin component. The first dosage form is structured, for example and without limitation, made using microencapsulation technology and/or other suitable controlled release technology, to provide a relatively slow rate of release of the atorvastatin component, and the second dosage form is structured, for example and without limitation, made using microencapsulation technology and/or other suitable controlled release technology, so that the atorvastatin component is released at a faster rate or over a shorter period of time relative to the atorvastatin release rate in the first dosage form. The niacin component is released at substantially equal rates or over substantially equal periods of time from both the first dosage form and the second dosage form.

One feature of this embodiment is that the atorvastatin component is provided to the subject at an effective therapeutically amount or concentration, for example and without limitation, a substantial or relatively large concentration, even a maximum concentration, for a period of time without an effective amount or concentration, e.g., without a therapeutically effective amount or concentration, of the niacin component being present, for example and without limitation, during the middle of the night, such as between about 9 pm or midnight to about 3 am or about 6 am to provide substantial benefits, for example, in reducing HDL and HDL-2b levels.

In one embodiment, the atorvastatin component may be released after the niacin component in both daily dosage forms, such as the dosage forms administered after lunch and after dinner, has been released and metabolized. Without wishing to limit the invention to any particular theory of operation, it is believed that this feature advantageously facilitates or even allows the atorvastatin component to change the LDL metabolism separately from the niacin component. This is beneficial since it is believed that both mechanisms working together tend to place stress on the HDL protein complex, which is the apoA-I supply that is often already reduced in many subjects or patients. ApoA-I is a primary component of HDL-2b, and has been measured and found to often be between 105-110 to 168. These apoA-I values are relatively low. The process of raising HDL-2b benefits from an increase in apoA-I before actually raising the HDL. By administering a dosage form or forms structured so that the niacin component has been released and metabolized before the atorvastatin component, it is believed that the apoA-I level is better maintained or increased relative to administering a dosage form or dosage forms having the same amounts of niacin component and atorvastatin component and structured to release these two components at the same time.

Thus, in one embodiment, the niacin component may be completely released about 4 to about 6, hours after the dosage form is administered after lunch and then after dinner. This niacin component release may be substantially completed when the atorvastatin component is released sufficiently to begin to lower the LDL level in the subject. The action of the niacin component, which is a hydrogen ion donor, is believed to clash or interfere with the lipid lowering and antioxidant effects of the atorvastatin component. An atorvastatin component, unlike other statins, is believed to have a LDL lowering effect and its metabolite is believed to have an anti-oxidant effect. In one embodiment, the atorvastatin component in the present dosage forms may be microencapsulated, for example and without limitation, using a pH controlled release mechanism, so that the atorvastatin component is released from the dosage form at two or more different rates, for example and without limitation, the first half of the atorvastatin component being released at a relatively fast or faster rate and, thereafter, the second half of the atorvastatin component being released at a relatively slow or slower rate.

In one embodiment, the dosage form may be structured so as to provide a microencapsulated atorvastatin component with a delayed release component, for example and without limitation, a coating substantially surrounding the microencapsulated atorvastatin component, for example and without limitation, an enteric coating, to delay the release of the atorvastatin component for a desired period of time, for example and without limitation, until the atorvastatin component is in the bowel, e.g., small bowel or intestine, of the subject to whom the dosage form is administered.

In one embodiment, the dosage form administered after lunch is structured so that the niacin component in the dosage form administered is released and substantially completes or exhausts its activity or action within about 5 hours after the dosage form is administered to a subject. The dosage form to be administered after dinner is similarly structured with regard to the niacin component. Both dosage forms are structured to delay the release of the atorvastatin component for about 10 hours after the dosage form is administered to the subject. In this embodiment, a major portion or amount of the action of the atorvastatin component occurs, such as in the middle of the night, after the action of the niacin component has been substantially completed.

In one embodiment, a dosage form to be taken after lunch is provided without an atorvastatin component and comprises a niacin component which is released for or over a period of about 4 hours to about 6 hours after the dosage form is administered to a subject. The dosage form to be taken after dinner is structured so that the niacin component is released for or over a period of about 4 hours to about 6 hours and an atorvastatin component is provided with the release of the atorvastatin component delayed for a period of about 8 hours to about 10 hours after the dosage form is administered to a subject.

In one embodiment, one or a single dosage form is administered daily to a subject, for example and without limitation, about 30 minutes to about 60 minutes after dinner. This dosage form comprises the entire daily doses of both a niacin component and an atorvastatin component. The dosage form is structured to release the niacin component for or over a period of about 4 hours to about 6 hours after the dosage form is administered to a subject; and to delay the release of the atorvastatin component for a period of about 6 hours to about 8 hours after the dosage form is administered to the subject. Substantially separate niacin component action and atorvastatin action is obtained using such a once daily dosage form.

Various embodiments and features of the present invention are described herein. Any feature or combination of features described herein are included within the scope of the present invention provided that the features included in any such combination are not mutually inconsistent as will be apparent from the context, this specification, and the knowledge of one of ordinary skill in the art. In addition, any feature or combination of features may be specifically excluded from any embodiment of the present invention.

Additional advantages and aspects of the present invention are apparent in the following detailed description, examples, and drawings.

### BRIEF DESCRIPTION OF THE DRAWINGS

FIGS. 1-12 are schematic drawings of different solid layered unit dosage forms in accordance with the present invention. Each of these dosage forms may be made using conventional/well known pharmaceutical manufacturing techniques, for example and without limitation, as disclosed elsewhere herein.

### DETAILED DESCRIPTION OF THE INVENTION

### Abbreviations and Definitions

An “active agent” or “active ingredient” is a component of a dosage form, pharmaceutical composition, or composition of the present invention that performs a biological function when administered or induces or affects (enhances or inhibits) a physiological process in some manner. “Activity” is the ability to perform the function, or to induce or affect the process. Active agents and ingredients are distinguishable from excipients such as carriers, vehicles, diluents, lubricants, binders, and other formulating aids, and encapsulating or otherwise protective components. Active ingredients may also be referred to herein as a “component” of the compositions of the present invention.

A “synergistic therapeutic HDL increasing effect,” or “synergistic therapeutic HDL-2b increasing effect,” as used herein, means that a given combination of at least 2 or 3 compounds exhibits synergy when tested in an HDL or HDL-2b increasing assay (see Assays for Testing the HDL or HDL-2b Increasing Activity, below). “Synergy,” as described, for example, by Chou, et al., Adv Enzyme Regul 22: 27-55 (1984), occurs when the measured effect (in this case, an HDL or HDL-2b increasing effect) of the compounds when administered in combination is greater than the additive effect of the compounds when each is administered alone as a single agent. Chou, et al. provide an exemplary method of measuring synergy based on Michaelis-Menton kinetics where combination effects are reduced to a numeric indicator referred to as the combination index (C.I.). Where the combination index is less than 1, synergism is indicated. Where the combination index is equal to 1, summation (also commonly referred to as additivity) is indicated. Where the combination index is greater than 1, antagonism is indicated.

The abbreviation “HDL” refers to high density lipoprotein. The abbreviation “HDL-2b” refers to the gradient gel electrophoresis subclass of HDL having the 2b designation, which includes apoprotein A-I.

The phrase “therapeutically effective amount” means an amount sufficient to produce a therapeutic result. Generally the therapeutic result is an objective or subjective improvement of a disease or condition, achieved by inducing or enhancing a physiological process, blocking or inhibiting a physiological process, or in general terms performing a biological function that helps in or contributes to the elimination or abatement of the disease or condition.

A “subject” as used herein generally refers to any living multicellular organism. Subjects include, but are not limited to humans, animals (e.g., cows, pigs, horses, sheep, dogs and cats) and plants, including without limitation hominoids (e.g., humans, chimpanzees, and monkeys). The term “patient” refers to both human and veterinary subjects.

The phrase “substantially homogeneous,” when used to describe a formulation (or portion of a formulation) that contains a combination of components, means that the components, although each may be in particle or powder form, are fully mixed so that the individual components are not divided into discrete layers or form concentration gradients within the formulation.

“Unit dosage form” refers to a composition intended for a single administration to treat a subject suffering from a disease or medical condition. Each unit dosage form typically comprises at least one active ingredient of this invention plus pharmaceutically acceptable excipients. Examples of unit dosage forms are individual tablets, individual capsules, bulk powders, and liquid solutions, emulsions or suspensions. Beneficial modification of the disease or condition may require periodic administration of unit dosage forms, for example, one or two unit dosage forms taken or administered one, two or more times a day, one or two with each meal or one or more meals, one or two every four hours or other interval, or only one per day. The expression “oral unit dosage form” indicates a unit dosage form designed to be taken orally. A “solid unit dosage form” indicates a unit dosage form in solid state at the time of administration.

“Controlled” or “sustained” or “time release” delivery are equivalent terms that describe the type of active agent delivery that occurs when the active agent is released from a delivery vehicle at an ascertainable and manipulatable rate over a period of time, which is generally on the order of minutes, hours or days, typically ranging from about thirty minutes or less to about 3 days, rather than being dispersed immediately upon entry into the digestive tract or upon contact with gastric fluid. A controlled release rate can vary as a function of a multiplicity of factors. Factors influencing the rate of delivery in controlled release include, without limitation, the particle size, composition, porosity, charge structure, and degree of hydration of the delivery vehicle and the active ingredients, the acidity of the environment (either internal or external to the delivery vehicle), and the solubility of the active agent in the physiological environment, i.e., the particular location along the digestive tract. “Intermediate time release” or “intermediate release” refers to those formulations that release active agent from the delivery vehicle over a period of less than 12 hours and more than 2 hours or 3 hours or 5 hours. In an exemplary embodiment, the period of release is from about 5 to 9 hours. In another exemplary embodiment, the period is from 5 to 8 hours. In another exemplary embodiment, the period is from 6 to 8 hours. In another exemplary embodiment, the period about 7 hours.

Components of the compositions of the invention may be present as the active compounds, pharmaceutically acceptable salts of the active compounds, substituted counterparts thereof, pharmaceutically acceptable equivalents thereof, precursors of the active compounds, prodrugs of the active compounds, metabolites, such as pharmaceutically active metabolites, of the active compounds, derivatives of the active compounds which provide the same or similar therapeutic activity or effectiveness as the active compounds, and the like and mixtures thereof. For example and without limitation, the atorvastatin component may be a combination of atorvastatin and one or more metabolites of atorvastatin. The ratio of atorvastatin to the one or more atorvastatin metabolites may be varied, as desired. The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al. Journal of Pharmaceutical Science 66: 1-19 (1977)). Certain specific compounds of the present invention contain both basic and acidic functionalities that allow the compounds to be converted into either base or acid addition salts.

The neutral forms of the components are preferably regenerated by contacting the salt with a base or acid and isolating the parent compound in the conventional manner. The parent form of the compound differs from the various salt forms in certain physical properties, such as solubility in polar solvents.

In addition to salt forms, the present invention may provide chemical compounds, such as niacin, statins, NSAIDs, and/or the pharmaceutical excipients, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment.

Certain chemical compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain chemical compounds of the present invention possess asymmetric carbon atoms (optical centers) or double bonds; the racemates, diastereomers, geometric isomers and individual isomers are encompassed within the scope of the present invention. In an exemplary embodiment, niacin is a racemate. In another exemplary embodiment, niacin is substantially (over 70%) enantiomerically pure in one of the stereoisomers.

The term “niacin,” as used herein, refers to nicotinic acid, nicotinic acid derivatives and prodrugs that function as adipocyte G-protein antagonists (e.g. acipimox), and all pharmaceutically acceptable equivalents and salts thereof (e.g. Niaspan®, Nicolar®, and the like) and mixtures thereof. See also U.S. Pat. No. 6,677,361; Miller et al., Am. J. Clin. Nutr. 8: 480-490 (1960); and Neuvonen et al., Br. J. Clin. Pharmacol. 32: 473-476 (1991), which are herein incorporated by reference in their entirety for all purposes). The term “nicotinic acid” refers to a pyridine-3-carboxylic acid (i.e. vitamin B₃), including its salts and/or pharmaceutically acceptable equivalents.

In an exemplary embodiment, the niacin component and/or the NSAID component is in powder form.

In some embodiments, the composition includes an intermediate release excipient, for example and without limitation, cellulosic intermediate release excipients, such as alkyl celluloses, hydroxyalkyl celluloses and the like and mixtures thereof, such as those materials sold under the trademark Methocel®. Other useful intermediate release excipients are discussed in detail elsewhere herein.

An intermediate release excipient may be present with, for example and without limitation, operatively coupled to, an active component, for example and without limitation, a niacin component, a NSAID component and/or an atorvastatin component. Such a intermediate release excipient allows an active component, for example and without limitation, the niacin component, the NSAID component and/or the atorvastatin component, to be released from the dosage form over a period of time, which is effective to facilitate reducing one or more of the side effects, such as flushing and risk of inflammation which often occur with niacin use.

In an exemplary embodiment, an intermediate release solid unit dosage form is provided. The intermediate release solid unit dosage form may comprise a niacin component, an atorvastatin component, and an intermediate release excipient, and may also include a delayed release component or excipient. The intermediate release excipient, and the niacin component, together with an optional non-steroidal anti-inflammatory drug (NSAID) component, may be present as a substantially homogeneous mixture and/or in a single layer of the unit dosage form. The NSAID component may be provided in an amount effective to reduce flushing in a subject or patient relative to the amount of flushing observed with the niacin component alone. The niacin component may be provided in an amount effective to increase HDL and/or HDL-2b levels and/or to increase the quality of the HDL and/or HDL-2b. In some embodiments, the niacin component and the NSAID component are provided in amounts effective to at least partially inhibit a prostaglandin or cyclooxygenase action.

The atorvastatin component may be present in an amount effective to reduce cholesterol levels. The delayed release component or excipient, if any, may be positioned or located to delay the release of the atorvastatin component from the dosage form so that the atorvastatin component is released from the dosage form after the niacin component has been released from the dosage form. This sequential release of the niacin component and the atorvastatin component, in accordance with the present invention, provides substantial benefits and treatment enhancements relative to releasing the niacin and atorvastatin substantially simultaneously, for example and without limitation, at substantially the same time after the dosage form is administered to a subject.

A intermediate release excipient may be operatively coupled to or otherwise provided or associated with the atorvastatin component so as to slow or otherwise control the release of the atorvastatin component from the dosage form, for example, relative to the release of an atorvastatin component from a substantially identical dosage form without the intermediate release excipient operatively coupled or otherwise provided or associated with to the atorvastatin component. Such controlled, for example and without limitation, slow, release, over a relatively extended period of time, of the atorvastatin component facilitates the effectiveness and/or efficiency of the atorvastatin component, for example and without limitation, by extending the time that effective concentrations of atorvastatin component are present in the subject or patient and/or by allowing reduced amounts of atorvastatin component to be present in the dosage form to obtain a substantially similar or same therapeutic effect, for example, relative to a substantially identical dosage form without the intermediate release excipient.

In another embodiment, the niacin component and the NSAID component may be present, for example and without limitation, in a single layer as a substantially homogeneous mixture. The mixture may be formed by thoroughly mixing the niacin component and the NSAID component. Methods of thoroughly mixing pharmaceutical agents are well known in the art and include, for example, automatic mixing methods, such as electronic rotating drum mixing.

Non-steroidal anti-inflammatory drug components (NSAIDs) at least partially inhibit the synthesis of prostaglandins, leukotrienes, and other compounds that are involved in the inflammatory process. In addition, they may protect the stomach lining, promote blood platelet formation, inhibit blood clotting, and regulate salt and fluid balance in the body. NSAIDs are effective in alleviating pain symptoms associated with ailments such as fever, arthritis, gout, bursitis, painful menstruation, and headache.

NSAIDS include aspirin as well as nonaspirin products. NSAIDs may be selected from: steroidal anti-inflammatory drugs including hydrocortisone and the like; antihistaminic drugs (e.g., chlorpheniramine, triprolidine); antitussive drugs (e.g., dextromethorphan, codeine, carmiphen and carbetapentane); antipruritic drugs (e.g., methidilizine and trimeprizine); anticholinergic drugs (e.g., scopolamine, atropine, homatropine, levodopa); anti-emetic and antinauseant drugs (e.g., cyclizine, meclizine, chlorpromazine, buclizine); anorexic drugs (e.g., benzphetamine, phentermine, chlorphentermine, fenfluramine); central stimulant drugs (e.g., amphetamine, methamphetamine, dextroamphetamine and methylphenidate); antiarrhythmic drugs (e.g., propanolol, procainamide, disopyraminde, quinidine, encamide); β-adrenergic blocker drugs (e.g., metoprolol, acebutolol, betaxolol, labetalol and timolol); cardiotonic drugs (e.g., milrinone, aminone and dobutamine); antihypertensive drugs (e.g., enalapril, clonidine, hydralazine, minoxidil, guanadrel, guanethidine); diuretic drugs (e.g., amiloride and hydrochlorothiazide); vasodilator drugs (e.g., diltazem, amiodarone, isosuprine, nylidrin, tolazoline and verapamil); vasoconstrictor drugs (e.g., dihydroergotamine, ergotamine and methylsergide); antiulcer drugs (e.g., ranitidine and cimetidine); anesthetic drugs (e.g., lidocaine, bupivacaine, chlorprocaine, dibucaine); antidepressant drugs (e.g., imipramine, desipramine, amitryptiline, nortryptiline); tranquilizer and sedative drugs (e.g., chlordiazepoxide, benacytyzine, benzquinamide, flurazapam, hydroxyzine, loxapine and promazine); antipsychotic drugs (e.g., chlorprothixene, fluphenazine, haloperidol, molindone, thioridazine and trifluoperazine); antimicrobial drugs (antibacterial, antifungal, antiprotozoal and antiviral drugs); propionic acid derivatives; acetic acid derivatives; fenamic acid derivatives; biphenylcarboxylic acid derivatives; and oxicams.

In an exemplary embodiment, the unit dosage form includes a NSAID selected from aspirin, ibuprofen, indomethacin, phenylbutazone, and naproxen. In another exemplary embodiment, the NSAID is aspirin.

The term “aspirin,” as used herein includes any appropriate form of acetylsalicylic acid including buffered aspirin, enteric coated aspirin, aspirin salts such as calcium acetylsalicylate, and mixtures of aspirin with acid acceptors.

It has been discovered that, surprisingly, an atorvastatin component and a niacin component can be administered to a subject together in a dosage form, for example, an unit dosage form, while reducing or even substantially eliminating the risk of the immediate and proximate disadvantageous interaction between the niacin component and the atorvastatin component. Ultimately, the subject to whom the dosage form is administered obtains enhanced benefits, for example and without limitation, increased amounts and/or quality of HDL and/or HDL-2b, from the combination of the niacin component and the atorvastatin component in the present dosage form relative to a dosage form containing the same amounts of these components in which the components are released from the dosage form at the same time.

Thus, the present invention may provide useful, and complementary benefits of both a niacin component and an atorvastatin component, for example, to control cholesterol levels in a subject, while reducing, or even substantially eliminating undesired effects when these two components are in a dosage form structured for simultaneous release of the niacin component and the atorvastatin component.

In one broad aspect of the invention, dosage forms, e.g., unit dosage forms are provided which comprise a therapeutically effective amount of a niacin component, and a therapeutically effective amount of an atorvastatin component. The dosage form is structured so that when the dosage form is administered to a subject, for example and without limitation, a human, the niacin component is released from the dosage form prior to the release of the atorvastatin component from the dosage form.

One embodiment of such unit dosage forms comprise: a therapeutically effective amount of a niacin component; a therapeutically effective amount of an atorvastatin component; and a delayed release component substantially surrounding the atorvastatin component and being effective to delay the release of the atorvastatin component from the dosage form. In a useful embodiment, the dosage form further comprises a controlled release excipient, for example and without limitation, an intermediate release excipient operatively coupled to, or otherwise provided or associated with the atorvastatin component and being present in an amount effective to slow or otherwise control the release of the atorvastatin component from the dosage form relative to a substantially identical dosage form without the controlled release excipient or component.

In one embodiment, the present invention provides the benefits and convenience of a unit dosage form, and the benefits of both a niacin component and an atorvastatin component. The unit dosage form allows both of these active components to be administered together to a subject and to be appropriately sequentially released from the dosage form to benefit the subject with reduced or little or substantially no undesirable interaction between the active components.

Without wishing to limit the present invention to any particular theory or mechanism of operation, it is believed that after the unit dosage form is administered, such as orally administered, to a subject, for example and without limitation, a human, the niacin component is released from the dosage form in the stomach or upper portion of the small bowel or intestine of the subject. The atorvastatin component, for example, coated with a delayed release component, such as an enteric component, passes to the small bowel unchanged. The delayed release component does not dissolve and expose or release the atorvastatin component, until it is well into the small bowel, for example, several hours after the niacin component is released from the dosage form. After the delayed release component is compromised, the atorvastatin component is released from the dosage form, for example and without limitation, is allowed to disperse in the body of the subject to whom the dosage form was administered. In the embodiment in which a controlled release excipient is operatively coupled to the atorvastatin component, the atorvastatin component, which may be present at a significantly lower dose, is released at a slower or a much slower rate, for example, relative to a substantially identical dosage form without the controlled release excipient.

In any event, the niacin component and the atorvastatin component are released from the dosage form at different times, and may be released at different locations, for example, in the gastrointestinal tract of the subject.

In one embodiment, the niacin component may advantageously be dosed twice a day, while the atorvastatin component may be dosed either once or twice a day. In one embodiment, both the niacin component and the atorvastatin component are dosed twice a day or only once a day. Since the dose of the atorvastatin component combined with the niacin component in a unit dosage form in accordance with the present invention is more effective, it may be desirable to dose the atorvastatin component twice a day, at a smaller daily dose, for example, relative to obtaining the same therapeutic effect while dosing the atorvastatin component, for example, at a larger daily dose, only once a day.

In one embodiment, the daily dosage of the atorvastatin component may be 5 mg (total 10 mg) twice a day, or 7.5 mg (total 15 mg) twice a day or 12.5 mg (total 25 mg), with each of these dosages being with 375 mg of niacin component, and 81 mg of aspirin component, if present. If the atorvastatin component is dosed once a day, the dose may be 10 mg or 15 mg or 25 mg or 35 mg daily, with each of these dosages being with 375 mg of niacin component, and as low as about 25 mg or 81 mg or more of aspirin component, if present.

In one embodiment, the present methods comprise administering a dosage form to a subject twice daily.

For example, the present methods may comprise administering a dosage form in accordance with the present invention about 30 to about 60 minutes after the subject has eaten lunch, that is the mid-day or about noon time meal; and administering a dosage form in accordance with the present invention to the subject about 30 to about 60 minutes after the subject has eaten dinner, that is the late afternoon or evening or last full meal of the day. In one embodiment, the total daily dose of the niacin component, for example, during the major portion, that is at least about 50%, of the time the subject is being administered dosage forms in accordance with the present invention or is being treated in accordance with a method of the present invention, is in a range of about 500 mg or about 750 mg to about 1000 mg or about 1500 mg.

In one embodiment, during the first or initial about three (3) to about ten (10) days, such as six (6) days, of treatment or administering the dosage forms, the amount of the niacin component in each dosage form, for example, taken twice a day, is in a range of about 62.5 mg to about 125 mg.

In one embodiment, during the next about five (5) to about twelve (12) days, such as eight (8) days, of treatment or administering the dosage forms, the amount of the niacin component in each dosage form, for example, taken twice a day, is about 200 mg to about 300 mg or about 400, for example and without limitation, about 250 mg. During the next about 12 to about 20 days, such as 16 days, of treatment or administering the dosage forms, the amount of the niacin component in each dosage form, for example, taken twice a day, is about 300 mg to about 500 mg or about 750 mg, for example and without limitation, about 375 mg.

In one embodiment, during the period after the first about 3 to about 10 days of treatment or administering the dosage forms, the dosage forms administered after lunch include about 250 mg of niacin component and the dosage forms administered after dinner include about 500 mg of niacin component.

In one embodiment, during the about 5 to about 12 days following the first about 3 to about 10 days of treatment, each dosage form administered after lunch includes about 250 mg of niacin component and each dosage form administered after dinner includes about 500 mg of niacin component. After the about 5 to about 12 day period of treatment or administering the dosage forms noted above, each dosage form administered in the next about 12 to about 20 days of treatment includes about 375 mg of niacin component, or dosage forms administered after lunch include about 250 mg of niacin component and dosage forms administered after dinner include about 500 mg of niacin component. Each dosage form administered in the time period thereafter, that is after the about 12 to about 20 day period noted above, may include about 750 mg of niacin component.

In an exemplary embodiment, the niacin component, or the combination of the niacin component and a NSAID component, is released to a subject for or over a period of about 6 hours or less. In one embodiment, the period is in a range of about 30 minutes or less to about 6 hours. In one embodiment, the dosage form may be structured to release about 10% per hour of the niacin component for the first about five (5) hours and about 40 to 50%, that is substantially the entire remainder, of the niacin component in the sixth (6th) hour.

In one aspect of the present invention, the niacin component may be replaced, in whole or in part, by a fibrate component. In an exemplary embodiment, the fibrate component is fenofibrate (C₂₀H₂₁ClO₄), including salts, prodrugs, pharmaceutically acceptable equivalents thereof and the like and mixtures thereof. If the niacin component is wholly replaced by a fibrate component, the composition advantageously does not include a NSAID component.

The fibrate component may be present in the present dosage forms in a therapeutically effective amount, for example and without limitation, in an amount in a range of about 67 to about 134 mg per dosage form. In one embodiment, for example and without limitation, when the fibrate component is used in conjunction with soy oil components and the like components and mixtures thereof, reduced amounts of the fibrate component, for example and without limitation, about 45 to about 84 mg per dosage form, may be employed.

Exemplary NSAID compounds and time periods for administration (or release from the dosage form) are as described in U.S. Patent Publication US 2005/0148556A1, the disclosure of which is hereby incorporated in its entirety herein by reference for all purposes.

Methods of assaying for HDL and/or HDL-2b levels are well known in the art. Typically, venous blood is drawn in the morning after an overnight fast. Blood for preparation of HDL GGE analysis may be drawn into ice-cooled disodium EDTA tubes. The major lipoprotein fractions are separated by a combination of ultracentrifugation and precipitation in accordance with the Lipid Research Clinics Protocol generally known in the art. Briefly, VLDL is separated from LDL and HDL by preparative ultracentrifugation. LDL and HDL are separated by precipitation of the LDL fraction with heparin/manganese. The LDL concentration is calculated by subtraction of the HDL portion from the total concentration before precipitation. HDL-3 is separated by ultracentrifugation at a density of 1.125 kg/L and HDL-2 cholesterol is calculated by subtracting the value of HDL-3 from that of total HDL. Cholesterol and triglyceride concentrations are determined in the VLDL, LDL, and HDL fractions. In each run, the cholesterol and triglyceride analyses may be standardized against two frozen control sera of different concentrations. The control sera may be double-checked against reference methods for cholesterol and triglyceride analyses for detection of possible drift in methodology or control sera over time.

Plasma apoA-I and B concentrations may be analyzed by competitive radioimmunoassay (Pharmacia Diagnostics AB).

HDL GGE subclasses may be analyzed by a modification of the technique described by Blanche et al., Biochim Biophys Acta. 665: 408-419 (1981). In short, HDL is separated as a plasma fraction within the densities of 1.070 and 1.21 kg/L and subject to electrophoresis on polyacrylamide gradient gels (PAA 4/30, Pharmacia). The proteins are stained with amido black and scanned at wavelength 570 nm. The absorption of the gel itself is subtracted from the curves of the HDL samples. The relative areas under the curve may be assessed. The absolute concentration in milligrams of protein per milliliter for each subclass may be derived by multiplying the relative estimates for the HDL GGE subclasses by the total protein concentration of the isolated HDL fraction. The protein concentration of HDL may be analyzed according to Lowry et al. J. Biol. Chem. 193: 265-275 (1951).

Alternatively, the serum sample is combined with a Direct HDL buffer so that lipoproteins other than HDL are selectively removed via a reaction with cholesterol esterase and cholesterol oxidase. Catalase is added to the buffer to remove the hydrogen peroxide by product without the formation of color. Catalase is inhibited with the addition of Direct HDL Activator and the remaining HDL cholesterol is specifically reacted with cholesterol esterase and cholesterol oxidase. In the presence of peroxidase, the peroxide end product reacts with a 4-aminoantipyrine and N-(2-hydroxy-3-sulfopropyl)-3,5-dimethoxyaniline to form a colored quinine dye, which is measured spectrophotometrically at 578 nm. The procedures may be performed using Direct HDL Reagent products from Elan Pharmaceuticals in conjunction with an ATAC® 8000 Random Access Chemistry System. with an ATAC® 8000 Random Access Chemistry System.

The following references provide further exemplary methods of measuring levels of HDL and/or HDL-2b: Lipid Research Clinics Program, Manual of Laboratory Operations, Lipid and Lipoprotein analysis, DHEW Publication NIH 75-628, Bethesda Md., National Institutes of Health (1982); Warnick et al., Clin Chem 31: 217-22 (1985); Sugiuchi et al., Clin Chem 41: 717-23 (1995); Johansson et al., Arteriosclerosis, Thrombosis, and Vascular Biology. 15: 1049-1056 (1995).

The unit dosage forms of the present invention may be provided as pharmaceutical compositions.

A variety of dosage forms are useful in administrating the compositions of the present invention, including oral dosage forms such as tablets, capsules, pills, powders, granules, elixirs, tinctures, suspensions, syrups, emulsions and the like.

The pharmaceutical preparation includes one or more dosage forms, e.g., unit dosage forms. The unit dosage form may be subdivided into unit doses containing appropriate quantities of the active ingredient(s). The unit dosage form can be a packaged preparation, the package containing discrete quantities of active ingredient, such as packeted tablets, capsules, powders in vials or ampoules, cachets, lozenges, or an appropriate number of any of these in packaged form. Unit dosage forms may be in a form suitable for oral, rectal, topical, intravenous injection or parenteral administration. Parenteral and intravenous forms can also include minerals and other materials to make them compatible with the type of injection or delivery system chosen.

Solid form preparations include, without limitation, powders, tablets, pills, capsules, cachets, suppositories, dispersible granules and the like. A solid unit dosage form is a unit dosage in solid form. Solid form may include solid carriers, which may also act as diluents, flavoring agents, binders, preservatives, tablet disintegrating agents, or an encapsulating material. A pharmaceutical composition of the present invention can be micronized or powdered so that it is more easily dispersed and solubilized by the body. Processes for grinding or pulverizing drugs are well known in the art, for example, by using a hammer mill or similar milling device. In powders, the carrier may be a finely divided solid, which is in a mixture with the finely divided active component. In tablets, the active ingredient may be mixed with the carrier having the necessary binding properties in suitable proportions and compacted in the shape and size desired.

Liquid form preparations include, without limitation, solutions, suspensions, and emulsions, for example, water or water/propylene glycol solutions, and the like. Aqueous solutions suitable for oral use can be prepared by dissolving the active component in water and adding suitable colorants, flavors, stabilizers, thickening agents and the like, as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, emulsions and the like. These preparations may contain, in addition to the active component, colorants, flavors, stabilizers, buffers, artificial and natural sweeteners, dispersants, thickeners, solubilizing agents, and the like.

Compositions of the present invention may also be administered as pharmaceutical compositions that include an intravenous (bolus or infusion), intraperitoneal, subcutaneous, and/or intramuscular dosage form.

The compositions of the present invention may be administered in admixture with suitable pharmaceutical diluents, extenders, excipients, or carriers (collectively referred to herein as a pharmaceutically acceptable carrier or carrier materials) suitably selected with respect to the intended form of administration and as consistent with conventional pharmaceutical practices. Similarly, cachets and lozenges are included.

The pharmaceutical compositions may also be administered alone or mixed with a pharmaceutically acceptable carrier. The carrier can be a solid or liquid, and the type of carrier is generally chosen based on the type of administration being used. Exemplary carrier includes, without limitation, lactose, agar, magnesium carbonate, magnesium stearate, talc, sugar, pectin, dextrin, starch, gelatin, tragacanth, methylcellulose, sodium carboxymethylcellulose, a low melting wax, cocoa butter, and the like. Specific examples of pharmaceutical acceptable carriers and excipients that can be used to formulate oral dosage forms of the present invention are well known to one skilled in the art. See, for example, U.S. Pat. No. 3,903,297, which is incorporated herein by reference in its entirety for all purposes.

Examples of pharmaceutical compositions useful in administering one or more components of the compositions disclosed herein are discussed, for example, in U.S. Pat. Nos. 3,845,770, 3,916,899, 4,034,758, 4,077,407, 4,777,049, 4,851,229, 4,783,337, 3,952,741, 5,178,867, 4,587,117, 4,522,625, 5,650,170, 4,892,739, and U.S. Patent Publication No. 2005/0148556 A1, all of which are incorporated herein by reference in their entireties for all purposes.

Further techniques and compositions for making dosage forms useful in the present invention are also well known to one skilled in the art. See, for example, 7 Modern Pharmaceutics, Chapters 9 and 10 (Banker & Rhodes, Eds., 1979); Pharmaceutical Dosage Forms: Tablets (Lieberman et al., 1981); Ansel, Introduction to Pharmaceutical Dosage Forms 2.sup.nd Ed. (1976); Remington's Pharmaceutical Sciences, 17.sup.th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers Therapeutic Applications: Drugs and the Pharmaceutical Sciences, Vol. 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), all of which are incorporated herein by reference in their entireties for all purposes.

Tablets can contain suitable binders, lubricants, disintegrating agents, coloring agents, flavoring agents, flow-inducing agents, and melting agents. For instance, without limitation, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes, and the like. Lubricants used in these dosage forms include, without limitation, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Pharmaceutical compositions may be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines.

Pharmaceutical compositions may also be coupled to soluble polymers as targetable drug carriers or as a prodrug. Suitable soluble polymers include, without limitation, polyvinylpyrrolidone, pyran copolymer, polyhydroxylpropylmethacry-lamide-phenol, polyhydroxyethylasparta-midephenol, and polyethyleneoxide-polylysine substituted with palmitoyl residues. Furthermore, an antineoplastic mitochondrial oxidant can be coupled to a class of biodegradable polymers useful in achieving controlled release of a drug, for example, polylactic acid (PLA), polyglycolic acid (PGA), copolymers of polylactic and polyglycolic acid (PLGA), polyepsilon caprolactone, polyhydroxy butyric acid, polyorthoesters, polyacetals, polydihydropyrans, polycyanoacylates, crosslinked or amphipathic block copolymers of hydrogels and the like and mixtures thereof.

Many biodegradable or bioerodable polymers, such as PLA, PGA, PLGA and the like and mixtures thereof, may be employed as controlled release components in accordance with the present invention.

Gelatin capsules can contain the active ingredient and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours, for example, for or over a desired period of time. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include, without limitation, solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example and without limitation, suitable solvents, preservatives, emulsifying agents, suspending agents, diluents, sweeteners, thickeners, melting agents and the like.

Liquid dosage forms for oral administration can contain coloring and flavoring to increase patient acceptance. For example and without limitation, water, a suitable oil, saline, aqueous dextrose (glucose), and related sugar solutions and glycols, such as propylene glycol, polyethylene glycols and the like, are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, ascorbic acid, and the like and mixtures thereof, are suitable stabilizing agents. Also used are citric acid and its salts and sodium EDTA. In addition, parenteral solutions can contain preservatives, such as benzalkonium chloride, methyl- or propyl-paraben, chlorobutanol and the like. Suitable pharmaceutical carriers are described in Remington's Pharmaceutical Sciences, Mack Publishing Company, a standard reference text in this field, which has been incorporated herein by reference in its entirety for all purposes.

Pharmaceutical compositions may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally or often be continuous rather than intermittent throughout the dosage regimen.

Pharmaceutical formulations may also include a suspending agent. Suspending agents are well known in the art and any appropriate suspending agent may be used with the compositions of the present invention. In an exemplary embodiment, the suspending agent is selected from methylcellulose and vegetable fiber, beeswax, carnauba wax, paraffin, and/or spermaceti, as well as synthetic waxes, hydrogenated vegetable oils, fatty acids, fatty alcohols, and the like and mixtures thereof.

In some embodiments, the pharmaceutical formulation and/or unit dosage form(s) include a controlled release component or excipient. Exemplary controlled release excipients include without limitation, arabic gum, agar, alginic acid, sodium alginate, bentonite, carbomer, sodium carboxymethylcellulose, carrageenan, powdered cellulose, cetyl alcohol, dioctyl sodium sulfosuccinate, gelatin, glyceryl monostearate, hydroxyethyl cellulose, hydroxypropyl cellulose, hydroxypropyl methylcellulose, methylcellulose, octoxynol 9, oleyl alcohol, polyvinyl alcohol, povidone, propylene glycol monostearate, sodium lauryl sulfate, sorbitan esters, stearic acid, stearyl alcohol, PGA, PLA, PLGA, tragacanth, and xanthan gum. In an exemplary embodiment, the controlled time release excipient is a methylcellulose. In another exemplary embodiment, the methylcellulose includes between about 40 percent and about 50 percent of the total weight of the pharmaceutical composition. Methylcelluloses may be obtained from several companies, including Dow Chemical under the trade name Methocel®.

High viscosity water-soluble 2-hydroxypropyl methyl cellulose (HPMC) may be useful in tablets and in the controlled-release tablet coating, due to its sustaining properties with respect to component release, such as niacin. High viscosity HMPC has a nominal viscosity, two percent solution, of about 100,000 CPS, methoxyl content of about 19-24, a hydroxypropyl content of about 7-12 percent, and a particle size where at least 90% passes through a USS 100 mesh screen (Methocel® K100MCR). Low viscosity HPMC may be used as the binder component of the tablet. An exemplary low viscosity HPMC has a methoxyl content of about 20-30%, a hydroxylpropyl content of about 7-12 percent, and a particle size where 100% will pass through a USS No. 30 mesh screen and 99% will pass through a USS 40 mesh screen (Methocel® EIS). In some cases, a portion of the high viscosity HPMC can be replaced by a medium viscosity HPMC, i.e., of about 2000-8,000 cps.

Useful hydrophobic components include natural and synthetic waxes such as beeswax, carnauba wax, paraffin, spermaceti, as well as synthetic waxes, hydrogenated vegetable oils, fatty acids, fatty alcohols and the like.

Coatings comprising a major portion of a polymeric material having a high degree of swelling on contact with water or other aqueous liquids may be used to further prolong the release of an active ingredient, such as niacin, from a tablet core. Such polymers include, inter alia, cross-linked sodium carboxymethylcellulose (Acdisol-FMC), cross-linked hydroxypropylcellulose, hydroxymethylpropylcellulose, e.g., Methocel® K15M, Dow Chem. Co., carboxymethylamide, potassium methylacrylate divinylbenzene copolymer, polymethyl methacrylate, cross-linked polyvinylpyrrolidine, high molecular weight polyvinylalcohol, and the like. Hydroxypropylmethyl cellulose is available in a variety of molecular weights/viscosity grades from Dow Chemical Co. under the Methocel® designation. See also, Alderman (U.S. Pat. No. 4,704,285). These polymers may be dissolved in suitable volatile solvents, along with dyes, lubricants, flavorings and the like, and coated onto the prolonged release tablets, e.g., in amounts equal to 0.1-5% of the total tablet weight, by methods well known to the art. For example, see Remington's Pharmaceutical Sciences, A. Osol, ed., Mack Publishing Co., Easton, Pa. (16th ed. 1980) at pages 1585-1593.

Enteric components or coatings can also be provided to the dosage forms to prevent release of the niacin component and/or the atorvastatin component until the dosage form reaches the intestinal tract, or even a specific region of the intestinal tract. For example, enteric components or coatings which act as a delayed release component for the atorvastatin component may have a composition or structure which will allow the release of the atorvastatin component only in the alkaline environment of the lower bowel. Such coatings comprise mixtures of fats and fatty acids, shellac and shellac derivatives and the cellulose acid phthalates, e.g., those having a free carboxyl consent of 9-15%. See, Remington's at page 1590, and Zeitova et al. (U.S. Pat. No. 4,432,966), for descriptions of suitable enteric coating compositions.

In an exemplary embodiment, the controlled release excipient is an intermediate release excipient. An intermediate release excipient is a controlled release excipient (discussed above) that is provided in sufficient amounts to allow administration of active ingredients over a period of less than about 12 hours and more than about 4 hours. In an exemplary embodiment, the period is from about 5 to 9 hours. In some embodiments, the administration of active ingredients is from about 5 to 8 hours or from about 6 to 8 hours. In another exemplary embodiment, the administration of active ingredients is approximately 7 hours.

Tablets may include in admixture, about 5-30% high viscosity hydroxypropyl methyl cellulose, about 2-15% of a water-soluble pharmaceutical binder, about 2-20% of a hydrophobic component such as a waxy material, e.g., a fatty acid, etc.

Useful controlled release excipients for use in tablets are disclosed, for example, in U.S. Pat. Nos. 5,126,145, 5,268,181, and U.S. Pat. No. 6,596,308, which are herein incorporated by reference in their entireties for all purposes.

Microencapsulation may be employed to couple or otherwise associate a controlled release excipient, an intermediate release excipient and/or a delayed release excipient to an active ingredient to achieve the desired rate of release of the active ingredient or the desired delay in releasing the active ingredient from the present dosage forms. In one embodiment, the material used to microencapsulate the active ingredient or ingredients may be biodegradable or bioerodable. Examples of such materials useful to delay and/or control the release of pharmaceuticals include materials which are conventional and/or well known in the art for such purpose or purposes. A number of such materials are set forth elsewhere herein.

Microencapsulation may involve one or more processes which are conventional and/or well known in the art. Such processes may include, without limitation, pan coating processing, air-suspension coating processing, centrifugal extrusion processing, vibrational nozzle processing, spray drying processing, interfacial polymerization, in-situ polymerization, matrix polymerization and the like. Examples of microencapsulation are set forth in Miller et al U.S. Pat. No. 6,399,096 and U.S. Patent Publication No. US 2007/0100015, and U.S. Patent Publication No. US 2008/0058292, each of which are incorporated herein by reference in their entireties for all purposes.

Determination of the proper dosage for a particular situation is within the skill of the practitioner. Generally, treatment is initiated with smaller dosages, which are less than the optimum dose of the compound. Thereafter, the dosage is increased by small increments until the optimum effect under the circumstances is reached. For convenience, the total daily dosage may be divided and administered in portions during the day.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the compositions (including pharmaceutical compositions) are equally applicable to the methods of treating disease states and/or the pharmaceutical compositions described herein.

All publications, patents, and patent applications cited herein are hereby incorporated herein by reference in their entireties for all purposes.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

Of course, each of the dosage forms shown may include one or more other ingredients, as disclosed elsewhere herein. These dosage forms are only illustrative of the present invention and are not to be considered to be limiting.

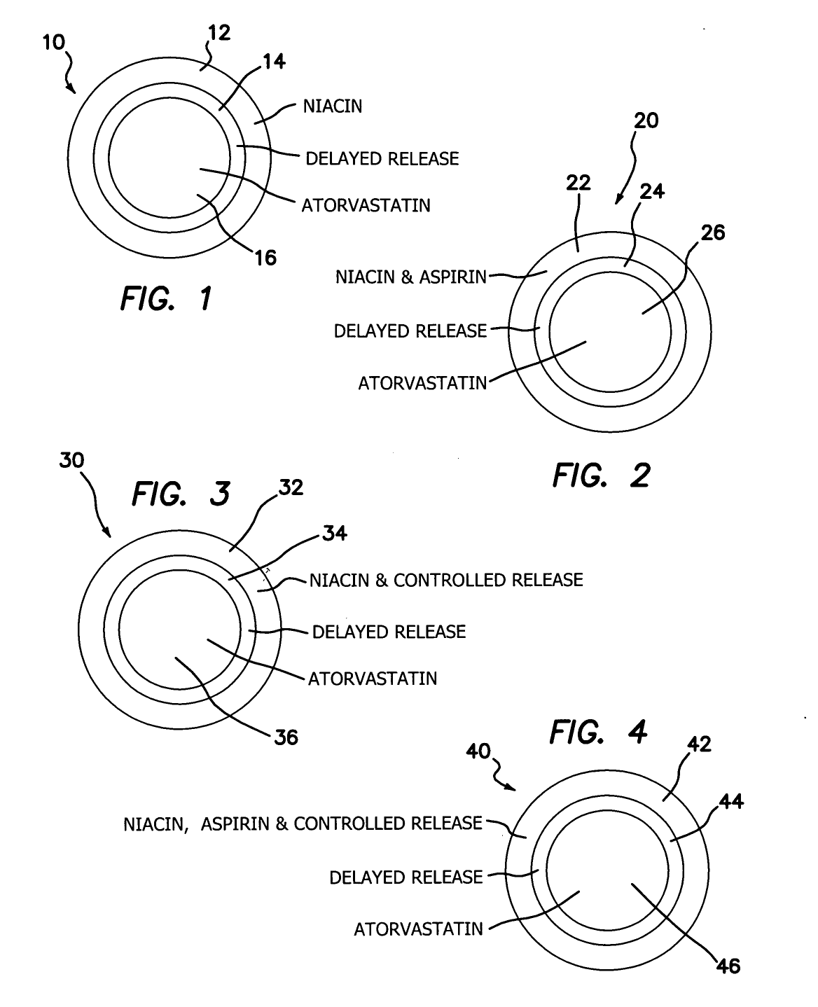

Each of the dosage forms illustrated in FIGS. 1-12 may include conventional and/or well known delayed release components, controlled release components, intermediate release excipients and one or more other ingredients commonly included in pharmaceuticals, for example, as described herein. Each of such dosage forms may be made using pharmaceutical manufacturing techniques, for example, as described herein, to achieve the release characteristics/profiles desired in accordance with the present invention, as described with regard to each of the individual dosage forms shown.

In addition, although each of the dosage forms in FIGS. 1-12 is illustrated as a layered structure, it should be noted that a layered structure is not required for the dosage forms in accordance with the present invention. The layered structures are provided and described for illustrative clarity and to make more easy an understanding of the present invention. As noted elsewhere herein, the dosage forms may be of any suitable physical structure effective to provide the desired component release characteristics/profiles in accordance with the present invention.

Now, with reference to the drawings, the dosage form 10, shown in FIG. 1, includes an outer layer 12 comprising niacin, an inner layer 14 comprising a delayed release component, such as an enteric component, and a core 16 comprising an atorvastatin component. The amounts of niacin, delayed release component and atorvastatin component are chosen as desired, for example, from amounts disclosed elsewhere herein, to provide the desired results.

When the dosage form 10 is orally administered to a human adult subject, the niacin in layer 12 is substantially immediately released from the dosage form. The delayed release component in layer 14 delays the release of the atorvastatin material in core 16 for a period of time, for example, about 6 hours, after the dosage form 10 is administered. After this about 6 hour delay period, the atorvastatin in core 16 is released from the dosage form 10 into the human adult subject to whom the dosage form 10 was administered.

The dosage form 20, shown in FIG. 2, is substantially similar to the dosage form 10. A primary difference is the presence of an aspirin component in outer layer 22.

Upon the oral administration of the dosage form 20, the niacin and aspirin in outer layer 22 are substantially immediately released from the dosage form. The aspirin is present in dosage form 20 as well as in other dosage forms illustrated in the drawings, in an amount, for example, as described elsewhere herein, to mitigate one or more of the side effects, such as flushing, which may occur because of the administration of niacin. The delayed release component in inner layer 24 delays the release of the atorvastatin component in core 26 by about 6 hours. After this about 6 hours delay period, the atorvastatin component in core 26 is released into the human adult subject to whom the dosage form 20 was administered.

The dosage form 30, shown in FIG. 3, is substantially similar to the dosage form 10. A primary difference is the presence of an intermediate release excipient or controlled release component in outer layer 32.

When dosage form 30 is orally administered to a human adult subject, the outer layer 32, because of the presence of the controlled release component, controllably releases the niacin from the dosage form over a period of about 5 hours. The delayed release component in inner layer 34 delays the release of the atorvastatin component in the core 36 by an additional about 6 hours delay period. After this additional 6 hours, the atorvastatin component in core 36 is released in the human subject to whom the dosage form 30 was administered.

The dosage form 40, shown in FIG. 4, is substantially similar to the dosage form 30. A primary difference is the presence of an aspirin component in outer layer 42.

The dosage form 40, after oral administration to a human adult, releases the niacin and aspirin component from outer layer 42 over a period of about 5 hours. The delayed release component in inner layer 44 delays the release of the atorvastatin component in core 46 by an additional about 6 hours. After such additional about 6 hours, the atorvastatin component is released into the human adult subject to whom the dosage form 40 was administered.

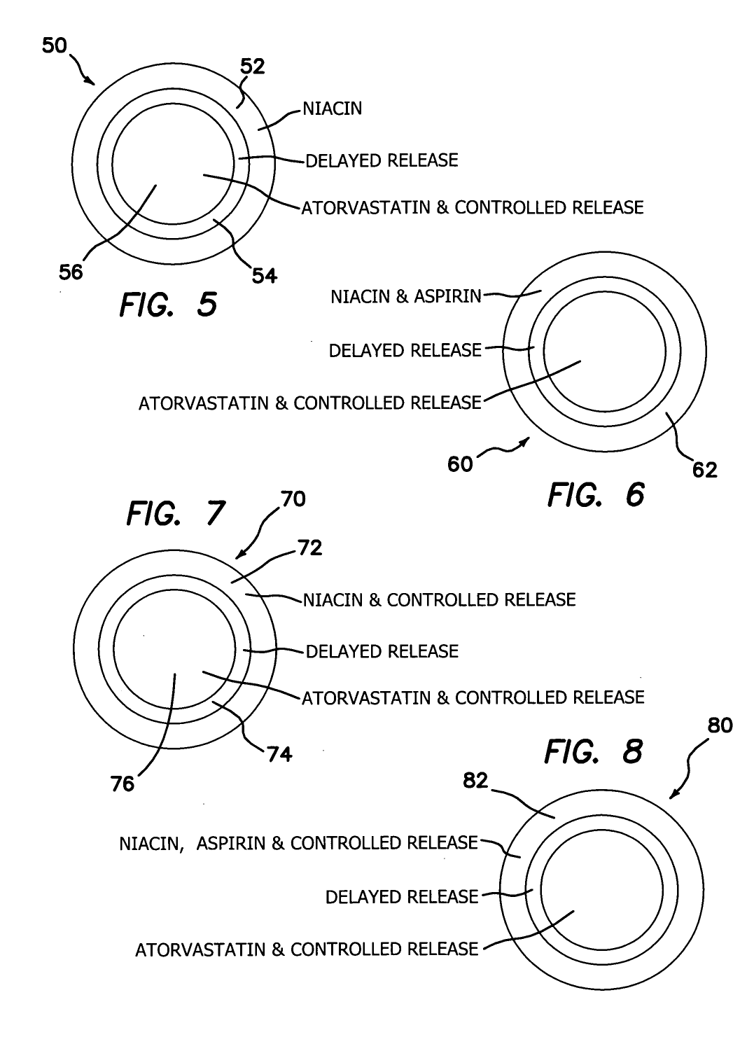

The dosage form 50, shown in FIG. 5, is substantially similar to the dosage form 10. A primary difference is the presence of a controlled release component in core 56 which acts to further control the release of the atorvastatin component in the core.

Upon oral administration of dosage form 50 to a human adult subject, the niacin in outer layer 52 is substantially immediately released from the dosage form into the subject. The delayed release component in inner layer 54 delays the release of the atorvastatin component from core 56 for an additional 6 hours. At the end of this additional 6 hours, the atorvastatin component is released at a rate controlled by the controlled release component so that over the next about 6 to about 8 hours all of the atorvastatin component from the core 56 is released into the body of the human subject to whom the dosage form 50 was administered.

The dosage form 60, shown in FIG. 6, is substantially similar to the dosage form 50. A primary different is the presence of a substantially uniform mixture of niacin and aspirin component in outer layer 62.

The dosage form 60, after oral administration to a human adult, releases the niacin and the aspirin component substantially immediately from the dosage form. The delayed release component in inner layer 54 delays the release of the atorvastatin component from core 56 for an additional 6 hours. At the end of this additional 6 hours, the atorvastatin component is released at a rate controlled by the controlled release component so that over the next about 6 to about 8 hours all of the atorvastatin component from the core 56 is released into the body of the human to whom the dosage form 50 was administered.

The dosage form 70, shown in FIG. 7, is substantially similar to the dosage form 50. A primary difference is the presence of a controlled release component or intermediate release excipient in outer layer 72.

Upon oral administration of the dosage form 70 to a human adult subject, the niacin in the outer layer 72 begins to be released from the dosage form 70 into the subject. The rate at which the niacin is released is controlled by the intermediate release excipient, for example, so that substantially all of the niacin is released from the outer layer 72 over a period of about 5 hours. The delayed release component in the inner layer 74 is such as to prevent substantially any release of the atorvastatin component in core 76 for at least 6 additional hours. After the about 6 additional hours, the atorvastatin component in core 76 is released at a controlled rate into the body of the human subject to whom the dosage form was administered. The controlled release component in core 76 may be such as to release the atorvastatin component over a period of about 4 to about 8 hours.

The dosage form 80, shown in FIG. 8, is substantially similar to the dosage form 70. A primary difference is that the dosage form 80 includes aspirin in the outer layer 82, together with niacin and a controlled release component or intermediate release excipient.

Upon oral administration of the dosage form 80 to a human adult subject, the niacin and aspirin component in the outer layer 82 begins to be released from the dosage form 80 into the subject. The rate at which the niacin and aspirin, which are provided in the outer layer 82 as a substantially homogeneous mixture, is controlled by the intermediate release excipient, for example, so that substantially all of the niacin and aspirin component are released from the outer layer 82 over a period of about 5 hours. The delayed release component in the inner layer 84 is such as to prevent substantially any release of the atorvastatin component in core 86 for at least about 6 additional hours. After the about 6 additional hours, the atorvastatin component in core 86 is released at a controlled rate into the body of the human subject to whom the dosage form 80 was administered. The controlled release component in core 86 may be such as to release the atorvastatin component over a period of about 4 to about 8 hours.

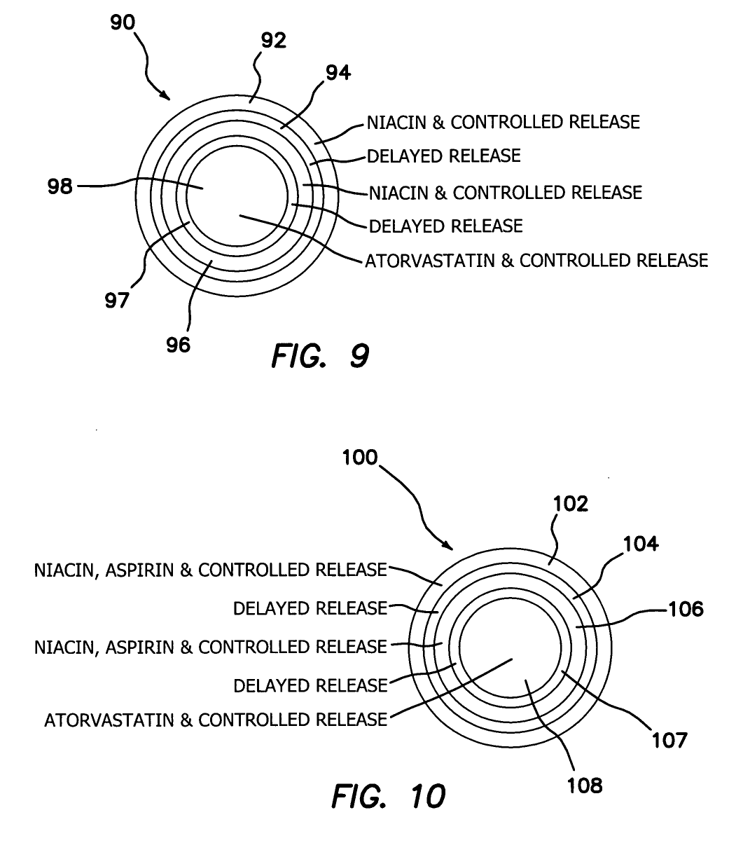

The dosage form 90, shown in FIG. 9, comprises an outer layer 92 comprising niacin and a controlled release component or intermediate release excipient. A first inner layer 94 comprises a delayed release component. A second inner layer 96 comprises an additional amount of niacin and a controlled release component or intermediate release excipient. A third inner layer 97 comprises a delayed release component. The core 98 of the dosage form 90 comprises an atorvastatin component and a controlled release component.

Upon administration of the dosage form 90 to a human adult subject, the niacin in the outer layer 92 is released over a period of time, controlled by the intermediate release excipient, for example, over about 5 hours. The delayed release component in the first inner layer 94 delays the release of the additional niacin in second inner layer 96 for a period of about 3 hours. At the end of this additional about 3 hours, the niacin in the second inner layer 96 is released over a period of time of about 3 hours, which is controlled by the intermediate release excipient in second inner layer 96. The delayed release component in third inner layer 97 delays the release of the atorvastatin component in core 98 for an additional about 6 hour period. After this additional about 6 hour period, the atorvastatin component in core 98 is released into the human subject to whom the dosage form 90 was administered over a period of about 6 to about 8 hours, which period is controlled by the controlled release component in core 98.

The dosage form 100, shown in FIG. 10, is substantially similar to the dosage form 90 in FIG. 9. A primary difference is that the outer layer 102 and the second inner layer 106 comprise an aspirin component in combination with the niacin and intermediate release excipient. The niacin and aspirin in both of these layers is provided in a substantially homogeneous mixture.

Upon administration of the dosage form 100 to a human adult subject, the niacin and aspirin in the outer layer 102 is released over a period of time, controlled by the intermediate release excipient, for example, over about 5 hours. The delayed release component in the first inner layer 104 delays the release of the additional niacin and aspirin component in second inner layer 106 for a period of about 3 hours. At the end of this additional about 3 hours, the niacin and aspirin in the second inner layer 106 is released over a period of time of about 3 hours, which is controlled by the intermediate release excipient in second inner layer 106. The delayed release component in third inner layer 107 delays the release of the atorvastatin component in core 108 for an additional about 6 hour period. After this additional about 6 hour period, the atorvastatin component in the core 108 is released into the human subject whom the dosage form 100 was administered over a period of about 6 to about 8 hours, which period is controlled by the controlled release component in core 108.

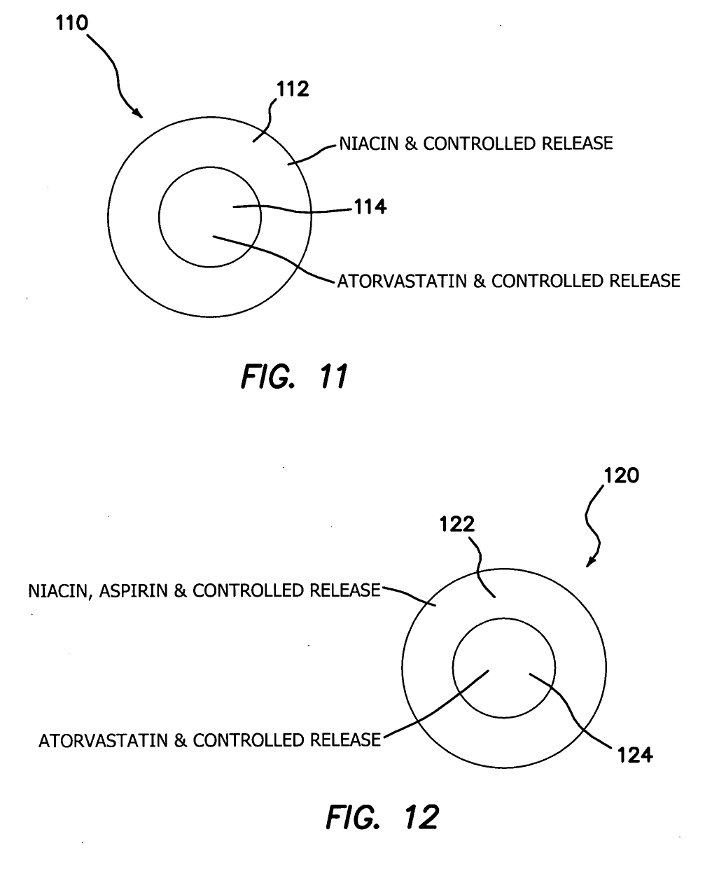

The dosage form 110, shown in FIG. 11, includes an outer layer 112 comprising niacin and a controlled release component or intermediate release excipient; and a core 114 comprising an atorvastatin component and a controlled release component.

Upon oral administration of the dosage form 110 to a human adult subject, the niacin in the outer layer 112 is released from the dosage form 110 over a period of time, for example, over about 5 hours, which time is controlled by the intermediate amount/composition of the intermediate release excipient. After this about 5 hour period, the atorvastatin component in the core 114 is released to the human subject to whom the dosage form 110 was administered over a period of time of an additional about 6 to about 10 hours, which time is controlled by the controlled release component in the core 114, for example and without limitation, by the composition/amount of the controlled release component.

The dosage form 120 shown in FIG. 12 is substantially similar to the dosage form 110. A primary difference is that the outer layer 122 includes an aspirin component in addition to the niacin and the controlled release component or intermediate release excipient.

Upon oral administration of the dosage form 120 to a human adult subject, the niacin and aspirin component, which are present in a substantially homogeneous mixture, in the outer layer 122 are released from the dosage form 120 over a period of time, for example, over about 5 hours, which time is controlled as noted in the discussion about with regard to the dosage form 110. After this about 5 hour period, the atorvastatin component in the core 124 is released to the human subject to whom the dosage form 120 was administered for or over a period of time of an additional about 6 to about 10 hours, which time is controlled by the controlled release component in the core 124 as discussed about with regard to core 114.

One or more of the unit dosage forms 10, 20, 30, 40, 50, 60, 70, 80, 90, 100, 110 and 120 may be used alone or in combination with one or more of the other of such unit dosage forms to treat a subject, such as a human subject, in accordance with the present invention, for example and without limitation, using the dosing regimens disclosed elsewhere herein, to obtain effective and useful treatment results as described elsewhere herein, such as increased levels of HDL and/or HDL-2b.

### Example 1

A dosage form 80, structured as shown in FIG. 8 is orally administered to a male human patient on a once daily basis for two (2) months. The patient's HDL level is measured both before starting such administering and after finishing such administering. It is found that the patient's HDL level has increased, for example, by about 12-14%, from the start to the finish of such administering.

### Comparative Example

Dosage forms are provided which are structured as shown in FIG. 8 except that the atorvastatin component and controlled release component are present in the outermost layer, and the niacin, aspirin and controlled release component are located in the central or core portion of the dosage form, which central portion in surrounded by the delayed release component. Such a dosage form is orally administered to a male human patient on a once daily basis for two (2) months. The patient's HDL level is measured both before starting such administering and after finishing such administering. It is found that the patient's HDL level has decreased, for example, by about 20%, from the start to the finish of such administering.

While this invention has been described with respect to various specific examples and embodiments, it is to be understood that the invention is not limited thereto and that it can be variously practiced within the scope of the following claims.

1. A unit dosage form comprising:
a therapeutically effective amount of a niacin component;
a therapeutically effective amount of an atorvastatin component; and
a delayed release component present in an amount effective to delay the release of the atorvastatin component from the dosage form.

2. The dosage form of claim 1, which is structured to release the niacin component from the dosage form prior to releasing the atorvastatin component from the dosage form.

3. The dosage form of claim 1, which further comprises a controlled release excipient operatively coupled to the atorvastatin component and being present in an amount effective to slow the release of the atorvastatin component from the dosage form relative to a substantially identical dosage form without the controlled release component.

4-6. (canceled)

7. The dosage form of claim 1, which further comprises a therapeutically effective amount of an additional niacin component and a second delayed release component positioned to delay the release of the additional niacin component from the dosage form relative to the release of the niacin component.

8. (canceled)

9. The dosage form of claim 1, wherein the delayed release component is an enteric component.

10. (canceled)

11. The dosage form of claim 1, which further comprises a therapeutically effective amount of a non-steroidal anti-inflammatory drug component.

12-13. (canceled)

14. The dosage form of claim 11, wherein the non-steroidal anti-inflammatory drug component comprises an aspirin component.

15-19. (canceled)

20. The dosage form of claim 1, which further comprises an intermediate release excipient component present in an amount and in a position effective to control the release of the niacin component.

21-24. (canceled)

25. The dosage form of claim 1, which is structured to release the niacin component into the body of a human subject to whom the dosage form has been orally administered by no more than about 6 hours after the dosage form is administered.

26-27. (canceled)

28. A unit dosage form comprising:
a therapeutically effective amount of a niacin component; and
a therapeutically effective amount of an atorvastatin component, the dosage form being structured so that, when the dosage form is administered to a subject, the niacin component is released from the dosage form prior to the release of the atorvastatin component from the dosage form.

29. (canceled)

30. The dosage form of claim 28, which further comprises a therapeutically effective amount of a non-steroidal anti-inflammatory drug component.

31. (canceled)

32. The dosage form of claim 28, wherein the non-steroidal anti-inflammatory drug component comprises an aspirin component.

33-35. (canceled)

36. The dosage form of claim 28, which is structured to release the niacin component from the dosage form over a first period of time after the dosage form is administered to a subject.

37. (canceled)

38. The dosage form of claim 36, which further comprises a therapeutically effective amount of a non-steroidal anti-inflammatory drug component, and is structured to release the non-steroidal anti-inflammatory drug component from the dosage form over a second period of time after the dosage form is administered to a subject.

39-48. (canceled)

49. The dosage form of claim 28, which is structured so that the release of the atorvastatin component from the dosage form, after the dosage form is administered to a subject, is delayed for at least about 4 hours and no more than about 14 hours.

50. (canceled)

51. A method of controlling cholesterol levels in a subject comprising administering to the subject an unit dosage form comprising a therapeutically effective amount of a niacin component, and a therapeutically effective amount of an atorvastatin component, the dosage form being structured so that after the administering step the niacin component is released from the dosage form prior to the atorvastatin component being released from the dosage form.

52. (canceled)

53. The method of claim 51, wherein the unit dosage form further comprises a therapeutically effective amount of a non-steroidal anti-inflammatory drug component, and is structured so that after the administering step the non-steroidal anti-inflammatory drug component is released from the dosage form prior to the atorvastatin component being released from the dosage form.

54-58. (canceled)

59. The method of claim 57, wherein the unit dosage form further comprises a therapeutically effective amount of a non-steroidal anti-inflammatory drug component, and is structured to release the non-steroidal anti-inflammatory drug component from the dosage form over a second period of time after the administering step.

60-65. (canceled)

66. The method of claim 51, wherein the unit dosage form is structured so that the release of the atorvastatin component from the dosage form after the administering step is delayed for at least about 4 hours and no more than about 14 hours.

67-69. (canceled)

70. The method of claim 51, wherein the administering step comprises administering the unit dosage form to the subject once daily.

71. The method of claim 51, wherein the administering step comprises administering an unit dosage form to the subject twice daily.

72. The method of claim 71, wherein an unit dosage form is administered to the subject about 30 to about 60 minutes after the subject has eaten lunch, and an unit dosage form is administered to the subject about 30 to about 60 minutes after the subject has eaten dinner.

73. (canceled)

74. The method of claim 72, wherein during the first about 3 to about 10 days of treatment each dosage form administered includes the niacin component in an amount in a range of about 62.5 mg to about 125 mg; during the next about 5 to about 12 days of treatment each dosage form administered includes the niacin component in an amount of about 200 to about 300 mg; and during the following about 12 to about 20 days of treatment each dosage form administered includes the niacin component in an amount of about 300 to about 1000 mg.

75-84. (canceled)
